(19) United States
(12) Patent Application Publication
(10) Pub. No.: US 2018/0216072 A1
(43) Pub. Date: Aug. 2, 2018

(54) BIOPRESERVED STEM CELLS ON MICROCARRIERS

(71) Applicant: ROOSTERBIO, INC., Frederick, MD (US)

(72) Inventors: Lye Theng Lock, Frederick, MD (US); Jonathan Rowley, Walkersville, MD (US)

(21) Appl. No.: 15/747,199

(22) PCT Filed: Jul. 25, 2016
(86) PCT No.: PCT/US2016/043839

§ 371 Date: Jan. 24, 2018

Related U.S. Application Data
(60) Provisional application No. 62/196,401, filed on Jul. 24, 2015.

(51) Int. Cl. C12N 5/0775; C12N 5/00

(52) U.S. Cl. CPC C12N 5/0663; C12N 5/0075; C12N 2531/00; C12N 5/0667; C12N 2500/62; C12N 5/0668

(57) ABSTRACT

Disclosed herein are compositions and methods involving stem cells biopreserved on microcarriers, which can be thawed and expanded, all while maintaining their key attributes, such as their proliferative capacity, identity, functionality, and potency. Disclosed is a method for generating microcarriers-seeded-stem cells, as well as a post biopreservation procedure for thawing and inoculating bioreactors to achieve a rapid and scalable stem cells expansion.

### BACKGROUND

Human stem cells characterized by their multi-lineage differentiation potential, and highly proliferative capacity are the most desired raw material in regenerative medicine. Typical cellular therapeutics require 50-100 million of cells per dose for treatment while an average of 600 million to 1 Billion cells are needed to generate a 1 cm³ block of cell mass/tissue in tissue engineering research. A good source of stem cells is not characterized only by their multi-lineage differentiation potential, but more importantly, their ability to be grown and expanded to larger cell quantities in a consistent manner A consistent and reproducible stem cells expansion method will ensure that cell therapy treatment can be offered at an affordable cost for patients, and that sufficient quantities of product can be made for commercialization and wide-spread use.

Today, the most widely used cell expansion platforms for stem cells culture are planar technologies such as flasks and multi-layer cell factories (J Rowley, E et al. BioProcess Int 10(3):7). In manufacturing scale-up production using planar systems, manufacturers can generate up to tens of billion of cells using multiple multilayer culture vessels, ran in parallel. The downside of this process is that they are very laborious, risky and the need for multiple robotic systems to aid in process manipulations can add significantly to the cost of the process. Furthermore, lot sizes are insufficient for a widely used commercial product.

Scalable technologies such as suspension bioreactors used routinely in protein, monoclonal antibodies and vaccine production, are proved to be a robust manufacturing platform. This technology operates in a closed and controlled environment which minimize the risk of contamination, and are shown to reduce the time, expense, and carbon footprint required for cell processing. More importantly, it is anticipated that such systems will allow one to reach a lot size of hundreds of billions to >1 trillion cells per manufacturing run, enabling commercially relevant lot sizes and with the economies of scale to reduce the cost per unit cells to achieve affordable therapeutics.

While bioreactor technology has been mostly used for suspension or non-adherent cell culture, stem cells, an inherently anchorage dependent cells will require a mean of surface such as microcarriers for their attachment and proliferation when grown in suspension. Microcarriers are small particles in the size range of 10-200s microns, with density similar to water, and are suspended easily when inoculated into a stirred suspension. Current process for adapting stem cell culture in suspension involve thawing cells out from cryopreserved condition, expanding them in flasks, and then seeding them onto microcarriers prior to inoculation into bioreactor (FIG. 1, top). There are a variety of seeding protocols available, and no standard procedures exist for such process which largely depends on the types of microcarriers used, the coatings on the microcarrier, as well as the bioreactors design and cell seeding/inoculation media. The efficiency of the seeding process is critical for establishing good expansion (cells must adhere to carrier to expand). Thus, the result in many microcarrier systems is a cell expansion and final cell yield which are often inconsistent and non-reproducible—most notably due to inconsistent seeding of carriers with cells.

### SUMMARY

Disclosed herein are compositions and methods involving stem cells biopreserved on microcarriers, which can be thawed and expanded, all while maintaining their key attributes, such as their proliferative capacity, identity, functionality, and potency. Disclosed is a method for generating microcarriers-seeded-stem cells, as well as a post biopreservation procedure for thawing and inoculating bioreactors to achieve a rapid and scalable stem cells expansion.

In particular, a composition is disclosed that comprises biopreserved stem cells adhered to a microcarrier. In some embodiments, the biopreserved stem cells have a cell viability of at least 70% and retain the ability to expand at least 10 fold after being stored for at least 6 months at −200 to −20° C. after recovery. For example, in these embodiments, the composition can comprise a cryopreservative agent, such as dimethyl sulfoxide (DMSO). In some embodiments, the biopreserved stem cells have a cell viability of at least 70% and retain the ability to expand at least 10 fold after being stored for 1 to 14 days at 0 to 10° C. after recovery. For example, in these embodiments, the composition can comprise a biopreservative agent, such as HypoThermosol®. In preferred embodiments, the biopreserved stem cells maintain cell surface marker expression, lineage differentiation potential, and cell functionality after recovery.

In some embodiments, the composition comprises about 1-50 cells per microcarrier. The cell-adhered microcarriers can be stored in container, such as a vial, bag, cryovial, or cryobag. In some embodiments, the biopreserved stem cells are present in this container at a concentration of 1×10³ to 5×10⁸ cells/ml.

In some embodiments, the stem cells comprise human pluripotent stem cells, such as embryonic stem cells or induced pluripotent stem cells. In some embodiments, the stem cells comprise human adult stem cells, such as bone marrow derived mesenchymal stem cell, adipose derived stem cells, umbilical cord derived stem cells, hematopoietic stem cells, endothelial stem cells, or multipotent progenitor cells.

Also disclosed is a method for expanding stem cells, comprising thawing and culturing the disclosed cell-adhered microcarriers in a suspension or perfusion bioreactor. The method can further involve culturing the cell-adhered microcarriers with cell-free microcarriers to provide additional surface area for the expanded cells. In some case, the cell-free microcarriers are provided in an amount effective to expand the cells to at least 200,000 cells/mL. For example, the cell-free microcarriers can be provided in an amount 2 to 10 fold greater than that of the cell-adhered microcarriers, i.e., providing 2 to 10 fold greater surface area. Again, the cells preferably maintain cell surface marker expression, lineage differentiation potential, and cell functionality after expansion.

Also disclosed is a method for preparing a bioink that involves thawing, recovering, and suspending in a viscous matrix stem cells from the disclosed cell-adhered microcarriers, or expanded from the disclosed cell-adhered microcarriers. In some cases, the suspended stem cells are at an average cell density of at least one million cells per milliliter. In some embodiments, the suspended stem cells are formed into aggregates containing on average at least 1,000 cells per aggregate (30-500 μm). In some cases, the viscous matrix comprises a biocompatible polymer, such as a biopolymer. For example, the polymer can be a polysaccharide, such as alginate.

Also disclosed is a cell expansion kit that contains the disclosed cell-adhered microcarrier composition in one container, and cell culture media and supplements for cell expansion. The kit can also contain a cell culture bioreactor or culture vessel for expanding the cells. In addition, the kit can contain cell-free microcarriers to provide additional surface area during expansion. The kit can further contain a harvest enzyme to harvest the cells from the microcarriers after expansion, along with a corresponding enzyme deactivation agent. Likewise, the kit can contain a filter or device to separate the cells from the microcarriers.

Also disclosed is a bioink kit that contains the disclosed cell-adhered microcarrier composition in one container, along with a biocompatible polymer and corresponding crosslinking agent. The kit can further contain cell culture media and supplements for cell expansion. The kit can further contain lineage specific differentiation media and supplements.

The details of one or more embodiments of the invention are set forth in the accompa-nying drawings and the description below. Other features, objects, and advantages of the invention will be apparent from the description and drawings, and from the claims.

### DESCRIPTION OF DRAWINGS

FIG. 1 (top) is a flow diagram of the current stem cell expansion process which involves thawing stem cells out of cryopreservation and seeding onto microcarriers prior to inoculation into bioreactor using different media or agitation rate. FIG. 1 (bottom) is the new product format of stem cells biopreserved on microcarriers, which allow direct inoculation of known amount of pre-seeded microcarriers into the bioreactor for a more consistent cell yield from the culture.

FIG. 2 are images of mesenchymal stem cells (arrow) attached and proliferating on microcarriers (A) pre- and (B) post-biopreservation.

FIG. 3 is an example of cell expansion profile on microcarriers pre- and post-biopreservation. Cells were seeded onto microcarriers and cultured for 3 days prior to collection for cell counts and biopreservation. On day 4, 8×10⁶ cells on microcarriers were thawed and seeded into bioreactor with additional fresh microcarriers for expansion. Cells were sampled after 3 days and data show they maintained similar proliferation capacity in suspension culture.

### DETAILED DESCRIPTION

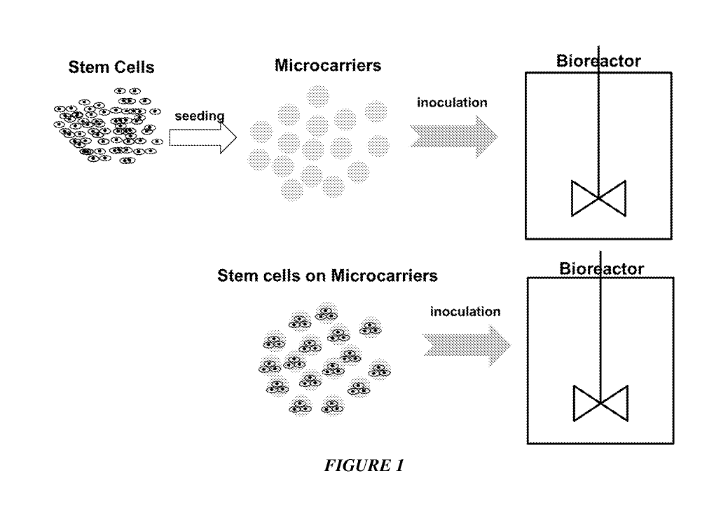

A failed batch of stem cells expansion in a clinical setting, can cost hundreds of thousands of dollars and have a major impact on the therapeutic supplies. Batch to batch inconsistency and unpredictability of stem cells expansion are mainly caused by the variability of initial cell attachment efficiency on microcarriers. This variability can be eliminated by using microcarriers with known amount of pre-seeded viable cells that are capable of expansion. To that end, disclosed herein are stem cells adhered onto microcarriers and biopreserved (FIG. 1, bottom), as a new format for attaining a consistent and scalable stem cell production process. This format removes the seeding step prior to inoculation, and allow users to inoculate a known amount of pre-seeded cells, to efficiently and consistently achieve the desired cell yield. Ultimately, this product configuration greatly simplifies and streamline stem cells production process, while minimizing risk and reducing the total cost by eliminating potential failed production batches.

A composition is disclosed that comprises biopreserved stem cells adhered to a microcarrier. The microcarriers containing cryopreserved cells can be ready to use after several days, weeks, or months of storage. The cell formulations allow for maintained cell viability and functionality. The cryopreserved cells can be stable for weeks to months or years, either in a frozen (i.e. cryopreserved) state. By frozen state, it is meant that the stable formulations are at or below a freezing temperature (e.g., at or below 0 degrees Celsius, and typically at least −20 degrees Celsius). In some embodiments, formulations stored in a frozen state comprise a cryopreservative agent.

In some embodiments, the biopreserved stem cells have a cell viability of at least 70% and retain the ability to expand at least 10 fold after being stored for at least 6 months at −200 to −20° C. after recovery. Therefore, in some embodiments, the compositions contain a cryopreservative agent. Non limiting examples of cryopreservative agents include dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, inorganic salts, or any combination thereof. In some cases, the cryopreservative contains CryoStor® CS2, CS5, or CS10 freeze media (BioLife Solutions, Inc) and 5% DMSO. In some cases, the cryopreservative contains between 1% and 15% DMSO, such as 2% to 7.5%, including 5% DMSO.

In some embodiments, the biopreserved stem cells have a cell viability of at least 70% and retain the ability to expand at least 10 fold after being stored for 1 to 14 days at 0 to 10° C. after recovery. Therefore, in some embodiments, the disclosed cell composition comprises a biopreservative agent, e.g., that scavenges free radicals, provides pH buffering, provides oncotic/osmotic support, contains energy substrates, has ionic concentrations that balance the intracellular state at low temperatures, or any combination thereof. For example, in some embodiments, the biopreservation formulation comprises HypoThermosol® (BioLife Solutions, Inc.), such as HypoThermosol®FRS. The components of HypoThermosol®FRS include Trolox (6-hydroxy-2,5,7,8-tetramethylchroman-2-carboxylic acid), Na⁺, K⁺, Ca²⁺, Mg²⁺, Cl⁻, H₂PO4⁻, HCO3⁻, HEPES, Lactobionate, Sucrose, Mannitol, Glucose, Dextran-40, Adenosine, and Glutathione, with a pH 7.6, and an osmolality of about 360. Therefore, in some embodiments, the disclosed cell composition comprises 2-10% DMSO and HypoThermosol® or HypoThermosol®FRS.

The term “cell” as used herein also refers to individual cells, cell lines, primary culture, or cultures derived from such cells unless specifically indicated. A “culture” refers to a composition comprising isolated cells of the same or a different type.

In some embodiments, the disclosed composition contains stem cells or progenitor cells. Pluripotential stem cells, adult stem cells, blastocyst-derived stem cells, gonadal ridge-derived stem cells, teratoma-derived stem cells, totipotent stem cells, multipotent stem cells, oncostatin-independent stem cell (OISCs), embryonic stem cells (ES), embryonic germ cells (EG), and embryonic carcinoma cells (EC) are all examples of stem cells. Stem cells can have a variety of different properties and categories of these properties. For example in some forms stem cells are capable of proliferating for at least 10, 15, 20, 30, or more passages in an undifferentiated state. In some forms the stem cells can proliferate for more than a year without differentiating. Stem cells can also maintain a normal karyotype while proliferating and/or differentiating. Stem cells can also be capable of retaining the ability to differentiate into mesoderm, endoderm, and ectoderm tissue, including germ cells, eggs and sperm. Some stem cells can also be cells capable of indefinite proliferation in vitro in an undifferentiated state. Some stem cells can also maintain a normal karyotype through prolonged culture. Some stem cells can maintain the potential to differentiate to derivatives of all three embryonic germ layers (endoderm, mesoderm, and ectoderm) even after prolonged culture. Some stem cells can form any cell type in the organism. Some stem cells can form embryoid bodies under certain conditions, such as growth on media which do not maintain undifferentiated growth. Some stem cells can form chimeras through fusion with a blastocyst, for example.

Some stem cells can be defined by a variety of markers. For example, some stem cells express alkaline phosphatase. Some stem cells express SSEA-1, SSEA-3, SSEA-4, TRA-1-60, and/or TRA-1-81. Some stem cells do not express SSEA-1, SSEA-3, SSEA-4, TRA-1-60, and/or TRA-1-81. Some stem cells express Oct 4, Sox2, and Nanog. It is understood that some stem cells will express these at the mRNA level, and still others will also express them at the protein level, on for example, the cell surface or within the cell.

In some embodiments, the disclosed composition comprises a cell other than a stem cell. The adult human body produces many different cell types. These different cell types include, but are not limited to, Keratinizing Epithelial Cells, Wet Stratified Barrier Epithelial Cells, Exocrine Secretory Epithelial Cells, Hormone Secreting Cells, Epithelial Absorptive Cells (Gut, Exocrine Glands and Urogenital Tract), Metabolism and Storage cells, Barrier Function Cells (Lung, Gut, Exocrine Glands and Urogenital Tract), Epithelial Cells Lining Closed Internal Body Cavities, Ciliated Cells with Propulsive Function, Extracellular Matrix Secretion Cells, Contractile Cells, Blood and Immune System Cells, Sensory Transducer Cells, Autonomic Neuron Cells, Sense Organ and Peripheral Neuron Supporting Cells, Central Nervous System Neurons and Glial Cells, Lens Cells, Pigment Cells, Germ Cells, and Nurse Cells.

Cells of the human body include Keratinizing Epithelial Cells, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet Stratified Barrier Epithelial Cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining bladder and urinary ducts), Exocrine Secretory Epithelial Cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), Clara cell of lung, Hormone Secreting Cells, Anterior pituitary cell secreting growth hormone, Anterior pituitary cell secreting follicle-stimulating hormone, Anterior pituitary cell secreting luteinizing hormone, Anterior pituitary cell secreting prolactin, Anterior pituitary cell secreting adrenocorticotropic hormone, Anterior pituitary cell secreting thyroid-stimulating hormone, Intermediate pituitary cell secreting melanocyte-stimulating hormone, Posterior pituitary cell secreting oxytocin, Posterior pituitary cell secreting vasopressin, Gut and respiratory tract cell secreting serotonin, Gut and respiratory tract cell secreting endorphin, Gut and respiratory tract cell secreting somatostatin, Gut and respiratory tract cell secreting gastrin, Gut and respiratory tract cell secreting secretin, Gut and respiratory tract cell secreting cholecystokinin, Gut and respiratory tract cell secreting insulin, Gut and respiratory tract cell secreting glucagon, Gut and respiratory tract cell secreting bombesin, Thyroid gland cell secreting thyroid hormone, Thyroid gland cell secreting calcitonin, Parathyroid gland cell secreting parathyroid hormone, Parathyroid gland oxyphil cell, Adrenal gland cell secreting epinephrine, Adrenal gland cell secreting norepinephrine, Adrenal gland cell secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, Mesangial cell of kidney, Epithelial Absorptive Cells (Gut, Exocrine Glands and Urogenital Tract), Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, Epididymal basal cell, Metabolism and Storage Cells, Hepatocyte (liver cell), White fat cell, Brown fat cell, Liver lipocyte, Barrier Function Cells (Lung, Gut, Exocrine Glands and Urogenital Tract), Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.), Epithelial Cells Lining Closed Internal Body Cavities, Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, Corneal endothelial cell, Ciliated Cells with Propulsive Function, Respiratory tract ciliated cell, Oviduct ciliated cell (in female), Uterine endometrial ciliated cell (in female), Rete testis cilated cell (in male), Ductulus efferens ciliated cell (in male), Ciliated ependymal cell of central nervous system (lining brain cavities), Extracellular Matrix Secretion Cells, Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Conical fibroblasts, Tendon fibroblasts, Bone marrow reticular tissue fibroblasts, Other (nonepithelial) fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Contractile Cells, Red skeletal muscle cell (slow), White skeletal muscle cell (fast), Intermediate skeletal muscle cell, Muscle spindle—nuclear bag cell, Muscle spindle—nuclear chain cell, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, Blood and Immune System Cells, Erythrocyte (red blood cell), Megakaryocyte, Monocyte, Connective tissue macrophage (various types), Epidermal Langerhans cell, Osteoclast (in bone), Dendritic cell (in lymphoid tissues), Microglial cell (in central nervous system), Neutrophil, Eosinophil, Basophil, Mast cell, Helper T lymphocyte cell, Suppressor T lymphocyte cell, Killer T lymphocyte cell, IgM B lymphocyte cell, IgG B lymphocyte cell, IgA B lymphocyte cell, IgE B lymphocyte cell, Killer cell, Stem cells and committed progenitors for the blood and immune system (various types), Sensory Transducer Cells, Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), Proprioceptive primary sensory neurons (various types), Autonomic Neuron Cells, Cholinergic neural cell (various types), Adrenergic neural cell (various types), Peptidergic neural cell (various types), Sense Organ and Peripheral Neuron Supporting Cells, Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), Enteric glial cell, Central Nervous System Neurons and Glial Cells, Neuron cell (large variety of types, still poorly classified), Astrocyte glial cell (various types), Oligodendrocyte glial cell, Lens Cells, Anterior lens epithelial cell, Crystallin-containing lens fiber cell, Pigment Cells, Melanocyte, Retinal pigmented epithelial cell, Germ Cells, Oogonium/oocyte, Spermatocyte, Spermatogonium cell (stem cell for spermatocyte), Nurse Cells, Ovarian follicle cell, Sertoli cell (in testis), and Thymus epithelial cell.

In some cases, the cells are mesenchymal stem cells (MSCs) or bone marrow stromal cells (BMSCs). These terms are used synonymously throughout herein. MSCs are of interest because they are easily isolated from a small aspirate of bone marrow, or other mesenchymal stem cell sources, and they readily generate single-cell derived colonies. Bone marrow cells may be obtained from iliac crest, femora, tibiae, spine, rib, knee or other mesenchymal tissues. Other sources of MSCs include embryonic yolk sac, placenta, umbilical cord, skin, fat, synovial tissue from joints, and blood. The presence of MSCs in culture colonies may be verified by specific cell surface markers which are identified with monoclonal antibodies. See U.S. Pat. Nos. 5,486,359 and 7,153,500. The single-cell derived colonies can be expanded through as many as 50 population doublings in about 10 weeks, and can differentiate into osteoblasts, adipocytes, chondrocytes (Friedenstein et al., 1970 Cell Tissue Kinet. 3:393-403; Castro-Malaspina, et al., 1980 Blood 56:289-301; Beresford et al., 1992 J. Cell Sci. 102:341-351; Prockop, 1997 Science 276:71-74), myocytes (Wakitani et al, 1995 Muscle Nerve 18:1417-1426), astrocytes, oligodendrocytes, and neurons (Azizi et al., 1998 Proc. Natl. Acad. Sci. USA 95:3908-3913); Kopen et al 1999 Proc. Natl. Acad. Sci. USA 96:10711-10716; Chopp et al., 2000 Neuroreport II 3001-3005; Woodbury et al., 2000 Neuroscience Res. 61:364-370). In rare instances, the cells can differentiate into cells of all three germlines. Thus, MSCs serve as progenitors for multiple mesenchymal cell lineages including bone, cartilage, ligament, tendon, adipose, muscle, cardiac tissue, stroma, dermis, and other connective tissues. See U.S. Pat. Nos. 6,387,369 and 7,101,704. For these reasons, MSCs currently are being tested for their potential use in cell and gene therapy of a number of human diseases (Horwitz et al., 1999 Nat. Med. 5:309-313; Caplan, et al. 2000 Clin. Orthoped. 379:567-570).

In some cases, MSCs can be defined by a variety of markers. For example, MSCs can be positive for CD73, CD90, CD166 and negative for CD14, CD34 and CD45.

The cells can be derived from a human or other animal. For example, cells can originate from a mouse, guinea pig, rat, cattle, horses, pigs, sheep, or goat. In some embodiments, the cells originate from non-human primates. In some cases, the cells are used as autologous or allogenic treatment.

In some cases, the cells are obtained from a cell culture, which involves population doubling (cell passages). In these cases, the cells are preferably from a population doubling less than or equal to 50, including a population doubling between 4 and 50, between 10 and 30. Therefore the cells can be from passage number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The compositions provided herein comprise cells that maintain high viability and functionality after extended storage. By viability it is meant that after preservation or storage, the cells are alive and capable of the same cell functions in existence prior to storage. In some cases, high viability means that at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the initial cell population is capable of survival, growth, and function after preservation or storage.

Cell viability can be determined using methods known in the art. A viability assay is an assay to determine the ability of organs, cells or tissues to maintain or recover viability. Viability can be distinguished from the all-or-nothing states of life and death by use of a quantifiable index between 0 and 1 (or 0 and 100%) (Pegg D E (1989). “Viability assays for preserved cells, tissues, and organs”. Cryobiology 26(3): 212-231). For example, examining the ratio of potassium to sodium in cells can serve as an index of viability. If the cells do not have high intracellular potassium and low intracellular sodium, then the cell membrane may not be intact, and/or (2) the sodium-potassium pump may not be operating well. Thus, many assays that measure cell membrane integrity are used as quantitiate measures of viability. These can be Trypan Blue, propidium iodide (PI), which are dyes that can penetrate leaky cell membranes and have been automated in commercially available cell counters. Other types of assays measure the overall metabolic rate of cell populations such as measuring total ATP, formazan-based assays (MTT/XTT) and Alomar blue-based or Resazurin-based assays. However quantitative measures of physiological function do not indicate whether damage repair and recovery is possible. An assay of the ability of a cell to adhere to a surface, spread and eventually migrate and divide may be more indicative of a live cell, but can make considerable more time and can be less quantitative. With that said, all of these tests can be used as viability assays to assess the success of cryopreservation techniques, the toxicity of substances, or the effectiveness of substances in mitigating effects of toxic substances.

In the disclosed studies, an automated PI membrane integrity assay (NucleoCounter NC100 by Chemometech Inc), was used as a fluorescent Live/Dead assay, and Alomar Blue was used to measure cell metabolism of aggregates. Functional tests were also used that show that the cells within aggregates can still adhere to cell culture plastic and grow out from the aggregate, and when the aggregates are maintained on non-adhesive surfaces the cells at the aggregate edge will join with other aggregates, creating fusion of aggregates into single, larger structures. It is the general assumption that many of the cell functions such as cytokine secretion, differentiation, and immunomodulatory function of hMSCs are maintained if the cells are still capable of adhering and spreading on culture plates, or if the cellular aggregates fuse (which is the 3D equivalent of adhering and spreading)

High viability of the cells (e.g., MSCs) in the compositions can be maintained as a result of the aqueous solutions in which the cells are stored. The aqueous solutions can, for example, comprise chemicals and nutrients that reduce cellular apoptosis and cellular death.

The compositions provided herein comprise cells at high volumes and high cell concentrations. In some embodiments, each of the disclosed microcarriers contain at least 1-50 cells. For example, in some embodiments, the composition comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90 or 100 cells.

The disclosed cryopreserved stem cells can be adhered to any microcarrier suitable for use in a microcarrier bioreactor system. The term “microcarrier” refers to a support matrix particle allowing for the growth of adherent cells in bioreactors. The disclosed microcarriers can be spheres, cylindricals or flat carriers. The microcarriers can be solid or porous. The microcarriers can have a diameter or size between 40-1000 μm.

Microcarriers can be made from a number of different materials. In some embodiments, the microcarriers are made with a non-biodegradable material, such as cellulose, DEAE-dextran, hydroxylated methacrylate, polyacrylamide, polystyrene, plastic, glass, ceramic, and silicone. In some embodiments, the microcarriers are made with a biodegradable materials such as collagen, alginate, dextran, gellan gum and gelatin. These microcarrier materials, along with different surface chemistries, can influence cellular behavior, including morphology and proliferation. Surface chemistries can include extracellular matrix proteins, recombinant proteins, peptides, and positively or negatively charged molecules. For example, the microcarriers can be coated with collagen, fibronectin, pronectin, matrigel, laminin, vitronectin, or e-cadherin. The microcarriers can be positively charged, or surface modified with peptide conjugate such as nanofiber or thermos reversible/responsive surface such as poly N-isopropylacrylamide (poly-NIPAAM). In some embodiments, the microcarriers are magnetic beads.

Microcarriers preferably have density that allows them to be maintained in suspension with gentle stirring. For example, the microcarriers can have a specific gravity between 1.015 g/cc and 1.035 g/cc. Microcarrier cell culture is typically carried out in spinner flasks, although other vessels such as rotating wall microgravity bioreactors or fluidized bed bioreactors can also support microcarrier-based cultures.

Several types of microcarriers are available commercially including alginate-based (GEM, Global Cell Solutions), dextran-based (Cytodex, GE Healthcare), collagen-based (Cultispher, Percell), and polystyrene-based (SoloHill Engineering) microcarriers. They differ in their porosity, specific gravity, optical properties, presence of animal components, and surface chemistries.

In some embodiments, the composition comprises about 1-50 cells per microcarrier. The cell-adhered microcarriers can be stored in container, such as a vial, bag, cryovial, or cryobag. In some embodiments, the biopreserved stem cells are present in this container at a concentration of 1×10³ to 5×10⁸ cells/ml.

Also disclosed is a method for expanding stem cells, comprising thawing and culturing the disclosed cell-adhered microcarriers in a suspension or perfusion bioreactor. The method can further involve culturing the cell-adhered microcarriers with cell-free microcarriers to provide additional surface area for the expanded cells. In some case, the cell-free microcarriers are provided in an amount effective to expand the cells to at least 200,000 cells/mL, including to about 200,000 to 1,000,000 cells/mL. For example, the cell-free microcarriers can be provided in an amount 2 to 10 fold greater than that of the cell-adhered microcarriers, i.e., providing 2 to 10 fold greater surface area. Again, the cells preferably maintain cell surface marker expression, lineage differentiation potential, and cell functionality after expansion.

Also disclosed is a method for preparing a bioink. As used herein, “bio-ink” means a liquid, semi-solid, or solid composition comprising a plurality of cells. In some embodiments, bio-ink comprises cell suspensions, cell aggregates, cell-comprising gels, multicellular bodies, or tissues. In some embodiments, the bio-ink additionally comprises support material. In some embodiments, the bio-ink additionally comprises non-cellular materials that provide specific biomechanical properties that enable bioprinting. In some cases, the viscous matrix comprises a biocompatible polymer, such as a biopolymer. For example, the polymer can be a polysaccharide, such as alginate.

In some embodiments, the microcarriers are a bioprintable material, such as a bioprintable hydrogel or alginate. In these embodiments, the microcarriers can be cultured in a bioreactor for expansion, collected, and directly loaded into cartridges as a bioink.

In some embodiments, preparing the bioink involves thawing, recovering, and suspending in a viscous matrix stem cells from the disclosed cell-adhered microcarriers, or expanded from the disclosed cell-adhered microcarriers. For example, the viscous solution can be a hydrogel, such as those described above. The viscosity is therefore preferably high enough to maintain uniform distribution while low enough to be suitable for extrusion by a bioprinter. In some embodiments, the bio-inks composition has a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. Viscosity can be measured by routine methods with a viscometer.

As used herein, “bioprinting” means utilizing three-dimensional, precise deposition of cells (e.g., cell solutions, cell-containing gels, cell suspensions, cell concentrations, multicellular aggregates, multicellular bodies, etc.) via methodology that is compatible with an automated, computer-aided, three-dimensional prototyping device (e.g., a bioprinter).

As used herein, “cartridge” means any object that is capable of receiving (and holding) a bio-ink or a support material.

In some embodiments, the container or cartridge has sterile ports or tubing that allows the cells and biomaterials to be expelled from the container or cartridge while maintain aseptic conditions where necessary.

In some embodiments, a bioprinter dispenses bio-ink from the cartridge in a specific pattern and at specific positions as directed by a computer aided design software in order to form a specific cellular construct, tissue, or organ. In order to fabricate complex tissue constructs, the bioprinter deposits the bio-ink at precise speeds and in uniform amounts. In some embodiments, a cartridge comprises one dispensing orifice. In various other embodiments, a cartridge comprises 2, 3, 4, 5, 6, 7, 8, 9, 10, 11, 12, 13, 14, 15, 20, 30, 40, 50, 60, 70, 80, 90, 100, or more dispensing orifices. In further embodiment, the edges of a dispensing orifice are smooth or substantially smooth.

Many types of cartridges are suitable for use with bioprinters. In some embodiments, a cartridge is compatible with bioprinting that involves extruding a semi-solid or solid bio-ink or a support material through one or more dispensing orifices. In some embodiments, a cartridge is compatible with bioprinting that involves dispensing a liquid or semi-solid cell solution, cell suspension, or cell concentration through one or more dispensing orifices. In some embodiments, a cartridge is compatible with non-continuous bioprinting. In some embodiments, a cartridge is compatible with continuous and/or substantially continuous bioprinting.

In some embodiments, a cartridge is a capillary tube or a micropipette. In some embodiments, a cartridge is a syringe or a needle. Many internal diameters are suitable for substantially round or cylindrical cartridges. In various embodiments, suitable internal diameters include, by way of non-limiting examples, 1, 10, 50, 100, 200, 300, 400, 500, 600, 700, 800, 900, 1000 or more μm, including increments therein. In other various embodiments, suitable internal diameters include, by way of non-limiting examples, 1, 2, 3, 4, 5, 6, 7, 8, 9, 10, 20, 30, 40, 50, 60, 70, 80, 90, 100 or more mm, including increments therein. In some embodiments, a cartridge has an internal diameter of about 1 μm to about 1000 μm. In a particular embodiment, a cartridge has an internal diameter of about 500 μm. In another particular embodiment, a cartridge has an internal diameter of about 250 μm. Many internal volumes are suitable for the cartridges disclosed herein. In various embodiments, suitable internal volumes include, by way of non-limiting examples, 0.1, 1, 10, 20, 30, 40, 50, 100, 200, 300, 400, 500, 600, 700, 800, 900, 1000 or more ml, including increments therein. In other various embodiments, suitable internal volumes include, by way of non-limiting examples, 1, 2, 3, 4, 5, 10, 20, 30, 40, 50, 60, 70, 80, 90, 100, 199, 300, 400, 500 or more ml, including increments therein.

In some embodiments, a cartridge is compatible with ink-jet printing of bio-ink and/or support material onto a receiving 2D or 3D surface such as that described in U.S. Pat. No. 7,051,654. In further embodiments, a cartridge includes dispensing orifices in the form of voltage-gated nozzles or needles under the control of the computer code described herein.

In some embodiments, a cartridge is marked to indicate the composition of its contents. In further embodiments, a cartridge is marked to indicate the composition of a bio-ink contained therein. In some embodiments, the surface of the cartridge is colored. In some embodiments, the outer surface of the cartridge is dyed, painted, marked with a pen, marked by a sticker, or a combination thereof.

In some cases, the cartridge is a single-use manifold system, such as that described in U.S. Pat. No. 6,712,963, which is disclosed herein for the teaching of single-use manifold units. Briefly, disposable tubing and flexible-wall containers can be assembled via aseptic connectors. These manifolds can interact with at least one remotely controlled pinch valve which engages only the outside surface of the manifold tubing. Such manifold and pinch valve systems can be used in conjunction with a peristaltic type of pump, which, together with the remotely operated pinch valve, can be operated by a controller which provides automated and accurate delivery of biotechnology fluid in an aseptic environment while avoiding or reducing cleaning and quality assurance procedures.

The disclosed cartridge is preferably configured to be filled with bioink aseptically and then protect the bioink from exposure to the environment to prevent contamination. Therefore, the cartridge preferably has a seal that maintains the closed system after being filled. The cartridge should also preferably be able to maintain cells in the bioink at a specific temperature. For example, the cartridge can be insulated.

The disclosed cartridge also preferably is configured to eject the bioink within. For example, the bioink can be ejected by air pressure, hydraulic pressure, screw driven pistons, or the like. As ejection can create significant pressures, the cartridge is preferably formed from a rigid material.

The cartridge has at least one orifice for ejection/dispersion of the bioink. However, multiple orifices can speed up printing. In some cases, the 3D printer is configured with two or more cartridges to dispense two or more types of cells. However, in some cases, a single cartridge contains two or more compartments and two or more orifices so as to dispense two types of cells at the same time. Alternatively, 2 or more cell types can be combined in cellular aggregates that are suspended in the biopolymer, or 2 cell types can be mixed together at an optimized ratio within the same hydrogel matrix and extruded at the same time.

In some embodiments, the cartridge contains a composition as disclosed herein containing cells suspended in a viscous matrix such that the cells are viable and ready for printing once removed from storage (e.g., frozen or non-frozen). In some cases, this means that the viscous matrix is sufficiently viscous to keep the cells uniformly dispersed in the composition, i.e., not settled to the bottom of the cartridge.

In some embodiments, the suspended stem cells are formed into aggregates containing on average at least 1,000 cells per aggregate (30-500 μm). In some cases, the suspended stem cells are at an average cell density of at least one million cells per milliliter.

Also disclosed is a cell expansion kit that contains the disclosed cell-adhered microcarrier composition in one container, and cell culture media and supplements for cell expansion. The kit can also contain a cell culture bioreactor or culture vessel for expanding the cells. In addition, the kit can contain cell-free microcarriers to provide additional surface area during expansion. The kit can further contain a harvest enzyme to harvest the cells from the microcarriers after expansion, along with a corresponding enzyme deactivation agent. Likewise, the kit can contain a filter or device to separate the cells from the microcarriers.

Also disclosed is a kit for producing bioink compositions. In some cases, the kit contains the disclosed cell-adhered microcarrier composition in one container, along with a biocompatible polymer and corresponding crosslinking agent. This agent can be in the same or different container as the cell-adhered microcarrier, so long as the biocompatible polymer and crosslinking agent are in different containers. The kit can further contain a means for mixing the ingredients of the kit, such as a syringe. The kit can further contain cell culture media and supplements for cell expansion. The kit can further contain lineage specific differentiation media and supplements.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

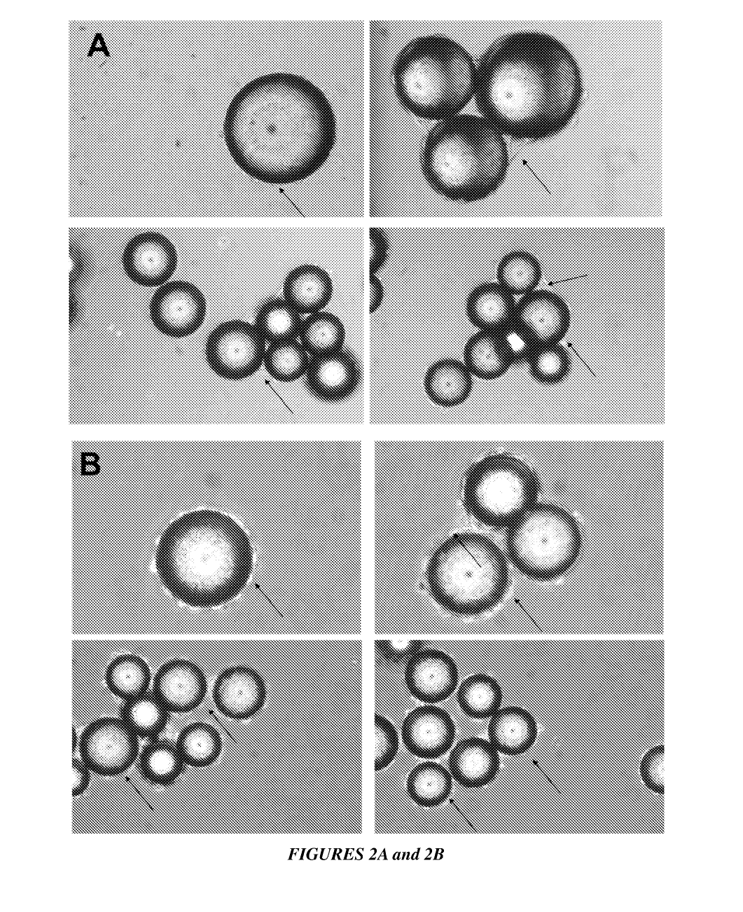

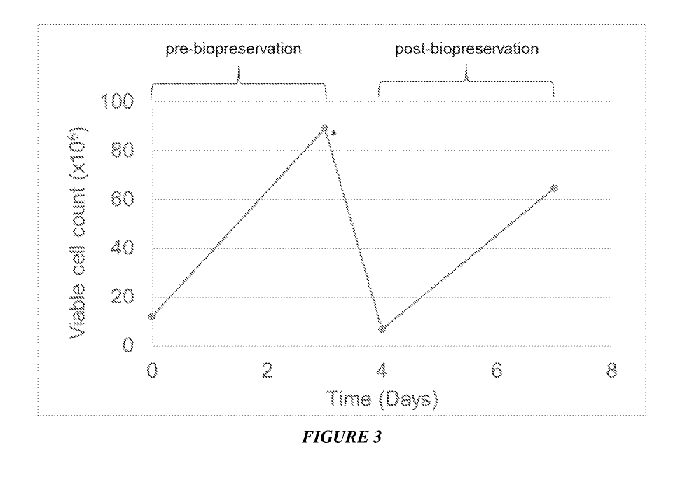

FIG. 2 shows human mesenchymal stem cells that were seeded onto microcarriers, cultured in suspension bioreactor, and then collected for cryopreservation within 3 days of culture. Upon thaw, consistent amount of hMSCs were successfully recovered from cryopreservation, with good cell viability. The hMSCs (arrow, FIG. 2) pre- and post-biopreservation, remain adhered onto the microcarriers and maintained similar morphology. The biopreserved cells on the microcarriers were further inoculated into suspension bioreactor culture, where fresh microcarriers were added to introduce additional surface areas for their expansion. As shown in FIG. 3, a 6-fold expansion was measured after 3 days of culture, similar to the growth rate of cells expansion prior to biopreservation step. This data indicate that hMSCs maintained their proliferative capacity after recovering from cryopreservation on microcarriers and they are capable of re-colonizing new surfaces for further expansion.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising biopreserved stem cells adhered to a microcarrier.

2. The composition of claim 1, wherein the biopreserved stem cells have a cell viability of at least 70% and retain the ability to expand at least 10 fold after being stored for at least 6 months at −200 to −20° C. after recovery.

3. The composition of claim 2, wherein the composition comprises a cryopreservative agent.

4. The composition of claim 3, wherein the cryopreservative agent comprises Dimethyl sulfoxide (DMSO).

5. The composition of claim 1, wherein the biopreserved stem cells have a cell viability of at least 70% and retain the ability to expand at least 10 fold after being stored for 1 to 14 days at 0 to 10° C. after recovery.

6. The composition of claim 5, wherein the composition comprises a biopreservative agent.

7. The composition of claim 6, wherein the biopreservative agent comprises HypoThermosol®.

8. The composition of claim 2, wherein the biopreserved stem cells maintain cell surface marker expression, lineage differentiation potential, and cell functionality after recovery.

9. The composition of claim 1, wherein the composition comprises about 1-50 cells per microcarrier.

10. The composition of claim 1, wherein the composition comprises the biopreserved stem cells at a concentration of 1×10³ to 5×10⁸ cells/ml.

11. The composition of claim 1, wherein cell-adhered microcarriers are stored in a vial, bag, cryovial, or cryobag.

12. The composition of claim 1, wherein the stem cells comprise human pluripotent stem cells.

13. The composition of claim 12, wherein the human pluripotent stem cells comprise embryonic stem cells or induced pluripotent stem cells.

14. The composition of claim 1, wherein the stem cells comprise human adult stem cells.

15. The composition of claim 14, wherein the human adult stem cells are selected from the group consisting of bone marrow derived mesenchymal stem cell, adipose derived stem cells, umbilical cord derived stem cells, hematopoietic stem cells, endothelial stem cells, and multipotent progenitor cells.

16. A method for expanding stem cells, comprising thawing and culturing the cell-adhered microcarriers of claim 1 in a suspension or perfusion bioreactor.

17. The method of claim 16, further comprising culturing the cell-adhered microcarriers with cell-free microcarriers.

18-26. (canceled)

27. A cell expansion kit comprising:
a) the composition of claim 1; and
b) cell culture media and supplements for cell expansion.

28. The kit of claim 27, further comprising a cell culture bioreactor or culture vessel.

29. The kit of claim 27, further comprising cell-free microcarriers.

30. The kit of claim 27, further comprising a harvest enzyme.

31. The kit of claim 27, further comprising an enzyme deactivation agent.

32. The kit of claim 27, further comprising a cell and bead separation filter or device.

33-35. (canceled)
